(19) United States
(12) Patent Application Publication
(10) Pub. No.: US 2013/0281524 A1
(43) Pub. Date: Oct. 24, 2013

(54) NOVEL TREATMENTS

(71) Applicants: Peter Blower, Essex (GB); Paul Sharpe, Essex (GB); Paul Durham, Springfield, MO (US)

(72) Inventors: Peter Blower, Essex (GB); Paul Sharpe, Essex (GB); Paul Durham, Springfield, MO (US)

(21) Appl. No.: 12/737,066

(22) PCT Filed: Jun. 4, 2009
(86) PCT No.: PCT/GB2009/050625

§ 371 Date: Jul. 19, 2011

(30) Foreign Application Priority Data
Jun. 5, 2008 (GB) 0810302.0
Oct. 29, 2008 (GB) 0819829.3
Oct. 29, 2008 (GB) 0819839.2

(51) Int. Cl. A61K 31/353

(52) U.S. Cl. CPC A61K 31/353
USPC 514/456; 549/399

(57) ABSTRACT

The present invention relates to the treatment or prevention of disorders where modulation of connexin 26 and/or p38 expression may be beneficial, and to tonabersat or an analogue of formula (I), and compositions comprising tonabersat or an analogue of formula (I) for use in said treatments.

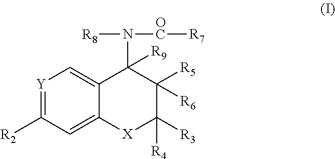

The present invention relates to the treatment or prevention of disorders where modulation of connexin 26 and/or p38 expression may be beneficial, and to tonabersat and its analogues of formula I below, and compositions comprising tonabersat or its analogues for use in said treatments.

International patent application WO 95/34545 discloses a series of specific named compounds, including tonabersat, otherwise known as cis-6-acetyl-4-(S)-(3-chloro-4-fluorobenzoylamino)-3,4-dihydro-2,2-dimethyl-2H-1-benzopyran-3-(S)-ol, which is a member of the class of drugs called neuronal gap junction blockers, and which is currently being investigated for a range of conditions including migraine, epilepsy, pain and other neurological conditions.

U.S. Pat. No. 5,948,811 (incorporated herein by way of reference) describes a class of compounds (‘the analogues of formula I’) which may be used for the prophylaxis and treatment of disorders within the central and peripheral nervous system, including migraine with or without aura.

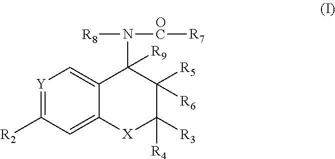

Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂—, CH(OH), SO₂, SO, CH₂—O—, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl.

Clinical trials (US Government Identifier NCT00311662) relate to the prophylaxis and treatment of migraine disorders including migraine with aura and migraine without aura by administration of tonabersat. Tonabersat is reported to demonstrate blockade of trigeminal parasympathetic reflexes (Parsons A, et. al. Tonabersat (SB-220453) a novel benzopyran with anticonvulsant properties attenuates trigeminal nerve-induced neurovascular reflexes. Brit. J. Pharmacol. 2001, 132, 1549-1557).

Connexin 26 is one of the connexin family members expressed in epidermis. It is not detected in normal and unlesioned skin but is expressed intensely at the cell periphery of keratinocytes in a psoriatic plaque. Dominant-acting missense mutations in connexin 26 result in five distinct skin disorders (Vohwinkel syndrome, keratitis-ichthyosis-deafness syndrome, palmoplantar keratoderma with deafness, hystrix-like ichthyosis with deafness, keratopachydermia and constrictions of fingers and toes with deafness), which share the common feature of hyperkeratosis. The dermatologic manifestation of human patients with connexin 26 mutations, together with the expression of connexin 26 in development, wound healing, and psoriasis, predict that the regulation of gap junctions is essential to normal epidermal differentiation (Djalilian A. R. et. al., Connexin 26 regulates epidermal barrier and wound remodelling and promotes psoriasiform response, J. Clin. Invest. 2006 May 1; 116(5), 1243-1253).

P38 is a serine kinase that plays a central role in inflammation (Schieven G L, Curr. Top. Med. Chem. 2005, 5, 921-928) and hence is considered a molecular target for classical inflammatory diseases (Kumar et. al., 2003, Nat. Rev. Drug Disc., 2, 717-726). Inflammatory mechanisms are believed to play a role in various psychiatric disorders and therefore, drugs acting to inhibit p38 or decreasing pro-inflammatory mediators are considered to have potential benefits in the treatment of depression, anxiety, schizophrenia, Alzheimer's and sleep disorders (See International Patent Application WO 2007/144390 and references cited therein).

There exists a need for safe, alternative and/or improved methods and compositions for the treatment or prevention of disorders where modulation of connexin 26 and/or p38 expression may be beneficial.

Temporomandibular joint disorder (TMD) is a collective term used to define pathological conditions involving temporomandibular joint, masticatory muscles and associated structures. Common symptoms of TMD include headache, jaw ache, earache neck pain, dizziness, tinnitus, and joint sounds.

There exists a need for safe, alternative and/or improved methods and compositions for the treatment or prevention of temporomandibular joint disorder (TMD) and associated symptoms, including headache, jaw ache, earache neck pain, dizziness, tinnitus, and joint sounds.

Trigeminal neuralgia is defined as inflammation of the trigeminal nerve (the fifth cranial nerve) that most commonly causes paroxysms of very intense lightning pain in the areas of the face the nerve supplies, including the lips, eyes, nose, scalp, forehead, gums, cheek, and chin. Trigeminal neuralgia may be associated with temporomandibular joint disorder (Paparo, F. et. al., European Review for Medical and Pharmacological Sciences, 2008, 12, 15 to 18). Trigeminal neuralgia may also be associated with intracranial tumors such as vestibular schwannoma (Bisi, M. A. et. al., Vestibular Schwannoma (Acoustic Neuroma) Mimicking Temporomandibular Disorders: A Case Report, Journal of Applied Oral Science, 2006, 14(6), 476 to 481).

There exists a need for safe, alternative and/or improved methods and compositions for the treatment or prevention of trigeminal neuralgia, which may or may not be connected to either TMD or intracranial tumors.

Connexin 26 is expressed in the cochlea and may play a role in K⁺ circulation between different partitions in the cochlea. Mutations in the gene for connexin 26 have been demonstrated to be linked to deafness in a large proportion of people (Gap Junction Channels Dysfunction in Deafness and Hearing Loss, Martinez A. D. et. al., Antioxidants and Redox Signalling, 2009, 11(2), 1-14). Tinnitus is defined as ringing in the ears or may be another noise that appears to originate from the head or ears. Tinnitus may be caused by ear infections, fluid in the ear, Meniere's syndrome, non-steroidal anti-inflammatory drugs, ear trauma from loud noises, or may even result from an aneurism or tumour on the acoustic nerve.

There exists a need for safe, alternative and/or improved methods and compositions for the treatment or prevention of hearing disorders and tinnitus, which may or may not be connected to TMD.

It is known that rhinitis, and in particular chronic rhinitis, may trigger a migraine with aura or without aura. However rhinitis and/or sinusitis are not necessarily present before, at the outset of or during the course of a migraine attack, and when present may worsen or improve independently of the migraine or any antimigraine agent which may be administered for the prophylaxis and/or treatment of the migraine.

Rhinitis, in particular chronic rhinitis, is not the only, or even the main, trigger for migraine, and there is no public evidence that, in verso, migraine with aura or without aura is a trigger or provides a stimulus for rhinitis or sinusitis in particular chronic rhinitis or sinusitis, or that administration of an antimigraine agent would effect prophylaxis and/or treatment of rhinitis and sinusitis in particular chronic rhinitis and/or sinusitis.

Rhinitis and sinusitis may be debilitating conditions which can typically result in impairment of the patient's inability to function normally in society, e.g. with problems with executive function, and may lead to ancillary disorders such as depression.

There exists a need for safe, alternative and/or improved methods and compositions for the treatment or prevention of rhinitis and sinusitis.

Cell bodies of trigeminal neurons that provide sensory innervation of the dura and nasal mucosa reside in the trigeminal ganglion in association with satellite glial cells where they communicate via gap junctions. Gap junctions, channels formed by connexins, modulate the excitability state of both neurons and glia under pathological conditions. Pre-treatment with tonabersat has been found to decrease gap junction communication and the level of connexin 26, and blocked p38 activation in both neurons and satellite glia.

Due to the ability to modulate, in particular, downregulate, connexin 26 and p38 expression, tonabersat or an analogue of formula I, may be useful in a variety of conditions where such modulation may be beneficial. Downregulation of connexin 26 and/or p38 expression in either the V1 and/or V2 regions of trigeminal ganglia may be particularly beneficial for the treatment of certain disorders.

In particular, the ability of tonabersat or an analogue of formula I, to prevent this sensitization and increased neuron-satellite glia signalling means that the drug may be particularly useful in the treatment or prevention of disorders such as rhinitis and sinusitis, temporomandibular joint disorder and associated symptoms, and hearing disorders and tinnitus that may or may not be connected to TMD.

Tonabersat or an analogue of formula I, may also be useful in the treatment of a variety of inflammatory disorders, including inflammatory skin disorders and wounds and also neurological and neuropsychiatric disorders where inflammation may play an active part.

Tonabersat or an analogue of formula I may also be useful in treating inflammatory hypersensitivity possibly mediated through Cx-26 gap junction-induced modulation (upregulation) of neural sensitivity. This neural hypersensitivity might well manifest as pain. Tonabersat appears to be effective in several models to reduce the hypersensitivity and may therefore be useful in treatment or prevention of inflammatory and/or neuropathic pain.

In a first aspect, the present invention provides for the use of tonabersat, or an analogue of formula I,

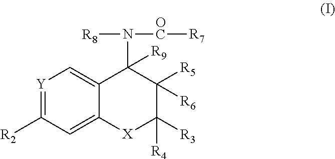

Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl or a pharmaceutically acceptable composition thereof, for the treatment or prevention of disorders where modulation of connexin 26 and/or p38 expression may be beneficial.

A preferred analogue of formula 1 is the compound carabersat or (trans-(+)-6-acetyl-4-(S)-(4-fluorobenzoylamino)-3,4-dihydro-2,2-dimethyl-2H-1-benzo[b]pyran-3R-ol, hemihydrate.

For therapeutic administration according to the present invention, tonabersat or a analogues of formula I, is most preferably employed in the form of its free base, but may also be used in the form of a pharmaceutically acceptable salt, preferably the hydrochloride salt. Alternative salts with pharmaceutically acceptable acids may also be utilised in prophylactic and/or therapeutic administration, for example salts derived from acids including, but not limited to, hydrobromic acid, phosphoric acid, acetic acid, fumaric acid, maleic acid, salicylic acid, citric acid, oxalic acid, lactic acid, malic acid, methanesulphonic acid and p-toluene sulphonic acid.

All references to tonabersat or an analogue of formula I, herein includes all pharmaceutically acceptable salts, and all solvates thereof.

For prophylactic and/or therapeutic administration according to the invention, tonabersat or an analogue of formula I, may be administered in pure form, but will preferably be formulated into any suitable pharmaceutically acceptable and effective composition which provides effective levels of the active ingredient in the body.

There is also provided as feature of the present invention, tonabersat or an analogue of formula 1 as described above, for use in the manufacture of a medicament for the treatment or prevention of a disorder where modulation of connexin 26 and/or p38 expression may be beneficial and any or all of the disorders listed below.

Accordingly, the present invention provides a method for the treatment or prevention of a disorder where modulation of connexin 26 and/or p38 expression may be beneficial, comprising administering to a patient in need thereof a pharmaceutically effective amount of tonabersat or an analogue of formula I, or a pharmaceutically acceptable composition thereof.

In another aspect, the present invention provides for tonabersat, or an analogue of formula I, or a pharmaceutically acceptable composition thereof, for use in the treatment or prevention of a disorder where modulation of connexin 26 and/or p38 expression may be beneficial.

In a preferred embodiment, the disorder is one where downregulation of connexin 26 and/or p38 expression may be beneficial.

In a more preferred embodiment, the disorder is one where downregulation of connexin 26 and/or p38 expression in either the V1 and/or V2 regions of trigeminal ganglia may be beneficial

In a further aspect, the present invention provides for the use of tonabersat or an analogue of formula I, or a pharmaceutically acceptable composition thereof, for the treatment or prevention of temporomandibular joint disorder and/or one or more associated symptoms.

Accordingly, the present invention provides a method for the treatment of temporomandibular joint disorder and/or one or more associated symptoms, comprising administering to a patient in need thereof a pharmaceutically effective amount of tonabersat or an analogue of formula I, or a pharmaceutically acceptable composition thereof.

In particular, the present invention provides a method for the treatment of temporomandibular joint disorder (TMD) and/or one or more associated symptoms of headache, jaw ache, earache neck pain, dizziness, tinnitus, or joint sounds, comprising administering to a patient in need thereof a pharmaceutically effective amount of tonabersat or an analogue of formula I, or a pharmaceutically acceptable composition thereof.

In another aspect, the present invention provides for tonabersat or an analogue of formula I, or a pharmaceutically acceptable composition thereof, for use in the treatment of temporomandibular joint disorder and/or one or more associated symptoms.

In particular, the present invention provides for tonabersat or an analogue of formula I, or a pharmaceutically acceptable composition thereof, for use in the treatment of temporomandibular joint disorder (TMD) and/or one or more associated symptoms of headache, jaw ache, earache neck pain, dizziness, tinnitus, or joint sounds.

In a further aspect, the present invention provides for the use of tonabersat or an analogue of formula I, or a pharmaceutically acceptable composition thereof, for the treatment or prevention of trigeminal neuralgia. In a preferred embodiment, the trigeminal neuralgia is associated with TMD. In another preferred embodiment, the trigeminal neuralgia is associated with an intracranial tumor, particularly vestibular schwannoma.

Accordingly, the present invention provides a method for the treatment of trigeminal neuralgia, comprising administering to a patient in need thereof a pharmaceutically effective amount of tonabersat or an analogue of formula I, or a pharmaceutically acceptable composition thereof.

In another aspect, the present invention provides for tonabersat or an analogue of formula I, or a pharmaceutically acceptable composition thereof, for use in the treatment of trigeminal neuralgia.

Tonabersat appears to be effective in several models to reduce hypersensitivity and may therefore be useful in treatment or prevention of inflammatory and/or neuropathic pain.

In a further aspect, the present invention therefore provides for tonabersat or an analogue of formula I, or a pharmaceutically acceptable composition thereof, for use in the treatment of trigeminal neuralgia, and other forms of pain such as pain associated with neuropathy or pain associated with inflammation.

Accordingly, the present invention provides a method for the treatment of pain associated with neuropathy or pain associated with inflammation, comprising administering to a patient in need thereof a pharmaceutically effective amount of tonabersat or an analogue of formula I, or a pharmaceutically acceptable composition thereof.

In a further aspect, the present invention provides for the use of tonabersat or an analogue of formula I, or a pharmaceutically acceptable composition thereof, for the treatment or prevention of hearing disorders or tinnitus. In a preferred embodiment, the tinnitus is associated with TMD.

Accordingly, the present invention provides a method for the treatment of hearing disorders or tinnitus, comprising administering to a patient in need thereof a pharmaceutically effective amount of tonabersat or an analogue of formula I, or a pharmaceutically acceptable composition thereof.

In another aspect, the present invention provides for tonabersat or an analogue of formula I, or a pharmaceutically acceptable composition thereof, for use in the treatment of hearing disorders or tinnitus.

In a further aspect, the present invention provides for the use of tonabersat or an analogue of formula I, or a pharmaceutically acceptable composition thereof, for the treatment or prevention of rhinitis or sinusitis. In a preferred embodiment, the rhinitis is chronic rhinitis.

Accordingly, the present invention provides a method for the treatment or prevention of rhinitis or sinusitis, comprising administering to a patient in need thereof a pharmaceutically effective amount of tonabersat or an analogue of formula I, or a pharmaceutically acceptable composition thereof.

In another aspect, the present invention provides for tonabersat or an analogue of formula I, or a pharmaceutically acceptable composition thereof, for use in the treatment or prevention of rhinitis or sinusitis, in particular chronic rhinitis.

The terms rhinitis and sinusitis, cover the full spectrum of rhinosinusitic disorders known to the skilled person and as listed in the World Health Organisation ICD Version 10 list. These include, but are not limited to, the following: Vasomotor rhinitis (J30.0), allergic rhinitis due to pollen (J30.1), other seasonal allergic rhinitis (J30.2), other allergic rhinitis (J30.3), unspecified allergic rhinitis (J30.4), chronic rhinitis (J31.0) (including Ozena, Rhinitis (NOS, atrophic, granulomatous, hypertrophic, obstructive, purulent, ulcerative)), chronic maxillary sinusitis (J32.0), chronic frontal sinusitis (J32.1), chronic ethmoidal sinusitis (J32.2), chronic sphenoidal sinusitis (J32.3), chronic pansinusitis (J32.4), other chronic sinusitis (J32.8), unspecified chronic sinusitis (J32.9).

In a further aspect, the present invention provides for the use of tonabersat or an analogue of formula I, or a pharmaceutically acceptable composition thereof, for the treatment or prevention of inflammatory disorders.

Accordingly, the present invention provides a method for the treatment or prevention of a disorder selected from inflammatory disorders, multiple myeloma, rheumatoid arthritis, pain, autoimmune disease, chronic inflammation, acute inflammation, hypersensitivity reaction, asthma, wound healing, tissue repair, skin disorders, psoriasis, hyperkeratosis disorders, such as Vohwinkel syndrome, keratitis-ichthyosis-deafness syndrome, palmoplantarkeratoderma with deafness, hystrix-like ichthyosis with deafness, keratopachydermia and constrictions of fingers and toes with deafness, depression, anxiety, schizophrenia, Alzheimer's and sleep disorders, comprising administering to a patient in need thereof a pharmaceutically effective amount of tonabersat or an analogue of formula I, or a pharmaceutically acceptable composition thereof.

In another aspect, the present invention provides for tonabersat or an analogue of formula I, or a pharmaceutically acceptable composition thereof, for use in the treatment or prevention of a disorder selected from inflammatory disorders, multiple myeloma, rheumatoid arthritis, pain, autoimmune disease, chronic inflammation, acute inflammation, hypersensitivity reaction, asthma, wound healing, tissue repair, skin disorders, psoriasis, hyperkeratosis disorders, such as Vohwinkel syndrome, keratitis-ichthyosis-deafness syndrome, palmoplantarkeratoderma with deafness, hystrix-like ichthyosis with deafness, keratopachydermia and constrictions of fingers and toes with deafness, depression, anxiety, schizophrenia, Alzheimer's and sleep disorders.

In a preferred embodiment the treatment of inflammatory skin disorders comprises topical administration of a composition comprising tonabersat or an analogue of formula I.

All treatments may be acute or prophylactic. For acute treatment, a rapid onset of action is preferred, and therefore, drugs that reach maximum plasma concentrations shortly after administration would be most beneficial. Accordingly, compositions providing rapid drug-release and/or dissolution are preferred.

Also included within the scope of the present invention are polymorphs, solvates and radiolabelled derivatives of tonabersat or an analogue of formula I, and pharmaceutically acceptable compositions thereof. References to tonabersat or an analogue of formula I, include such polymorphs, solvates and radiolabelled derivatives thereof.

Tonabersat or an analogue of formula I, may be delivered alone, but will generally be delivered in the form of a pharmaceutically acceptable composition thereof, which comprises tonabersat or an analogue of formula I, and one or more pharmaceutically acceptable diluents or carriers selected with regard to the intended route of administration.

Treatment with tonabersat or an analogue of formula I, or a pharmaceutically acceptable composition thereof, may be conducted at a unit dose of between 1 to 1000 mg, suitably 1 to 500 mg, for example an amount in the range of from 2 to 400 mg such as 2, 5, 10, 20, 30, 40, 50, 80, 100, 200, 300 and 400 mg of the active compound.

Unit doses will normally be administered once or more than once per day, for example 1, 2, 3, 4, 5 or 6 times a day, more usually 1 to 4 times a day, such that the total daily dose is normally in the range, for a 70 kg adult of 1 to 1000 mg, for example 1 to 500 mg, that is in the range of approximately 0.01 to 15 mg/kg/day, more usually 0.1 to 6 mg/kg/day, for example 1 to 6 mg/kg/day.

Preferably, the tonabersat or an analogue of formula I, or a pharmaceutically acceptable salt thereof, is administered to the patient at dose ranges of approximately 0.01 to 15 mg/kg/day, more usually 0.1 to 6 mg/kg/day, for example 1 to 6 mg/kg/day.

It is preferred that tonabersat or an analogue of formula I, is administered in the form of a pharmaceutical composition, such as a composition for oral, including sub-lingual, intranasal, rectal, topical, parenteral (especially intravenous), ocular or aural administration.

Pharmaceutical compositions suitable for the delivery of tonabersat or an analogue of formula I, and methods for their preparation will be readily apparent to those skilled in the art. Such compositions and methods for their preparation may be found, for example, in Remington's Pharmaceutical Sciences, 19th Edition (Mack Publishing Company, 1995).

Compositions suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations. Liquid formulations include suspensions, solutions, syrups and elixirs. Liquid formulations may also be prepared by the reconstitution of a solid, for example, from a sachet.

Orally administrable compositions may be in the form of oral solid compositions, such as tablets, capsules, pastilles, pellets, pills, lozenges powders and granules. The composition may be in solid form which melts on contact with the tongue of the patient, for example in the form of disintegrating tablets sold under the trade name ZYDIS®. Shaped oral compositions are preferred, since they are more convenient for general use.

Solid forms for oral administration are usually presented in a unit dose, and contain conventional additives such as adjuvants, binding agents, diluents, disintegrants, dispersing agents, excipients, fillers, tabletting agents, lubricants, colorants, flavourings, desiccants, humectants, and wetting agents.

Pills, pellets and tablets may be coated according to well known methods in the art. Oral solid formulations also include conventional sustained release formulations, such as tablets or granules having an enteric coating.

Suitable fillers include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

Solid oral compositions are prepared by admixture, and may be prepared by conventional methods of blending, filling, tabletting or the like. Repeated blending operations may be used to distribute the active agent throughout those compositions employing large quantities of fillers. Such operations are, of course, conventional in the art.

They may also be in the form of oral fluid preparations, including liquid preparations, such as aqueous or oily blends, mixtures, suspensions, solutions, emulsions, syrups, tinctures and elixirs, and gel preparations.

They may also be presented as dilutable fluid concentrates or dry product reconstitutable powders for dilution or reconstitution with water or other suitable vehicle before use.

Oral fluid preparations, including gels and liquid preparations may contain conventional additives such as: suspending agents, for example sorbitol, syrup, methyl cellulose, gelatin, hydroxyethylcellulose, carboxymethyl cellulose, aluminium stearate gel or hydrogenated edible fats; emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid; and, if desired, conventional flavouring or colouring agents.

Compositions for oral administration may be formulated to be immediate and/or modified release. Modified release formulations include delayed-, sustained-, pulsed-, controlled-, targeted and programmed release.

Compositions suitable for parenteral administration include injectable and infusible aqueous or oily blends, mixtures, suspensions, solutions, emulsions and low-viscosity gel preparations. Compositions for parenteral administration may be formulated to be immediate and/or modified release. Modified release formulations include delayed-, sustained-, pulsed-, controlled-, targeted and programmed release.

The parenteral compositions for use in the invention may be prepared as long acting depot preparations. Such formulations may be administered by intramuscular injection. Thus, for example, the composition of the invention may be formulated with suitable polymeric or hydrophobic materials (for example as an emulsion in a pharmaceutically acceptable oil) or ion exchange resins, or as sparingly soluble derivatives, for example, as a sparingly soluble salt.

Advantageously, adjuvants such as a local anaesthetic, preservatives and buffering agents are also dissolved, emulsified or suspended in the vehicle.

Such compositions are prepared by admixture of the compound and a solvent or vehicle. The compound, depending on the vehicle and the concentration, can be emulsified, suspended or dissolved. Parenteral compositions are normally prepared by with the compound and a vehicle which is sterile, and/or the composition is sterilised, before filling into a suitable vial or ampoule and sealing.

To enhance the stability, the composition may also be presented as a dry product reconstitutable powder for reconstitution with water or other suitable vehicle before use. A fluid composition can be frozen after filling into the vial and freeze-dried under vacuum.

Tonabersat or an analogue of formula I, may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™ Bioject™, etc.) injection.

Formulations for topical administration may be formulated to be immediate and/or modified release. Modified release formulations include delayed-, sustained-, pulsed-, controlled-, targeted and programmed release.

Compositions for use in surgical wounds may be prepared as long acting depot preparations. Such formulations may be administered by implantation (for example subcutaneously or intramuscularly) or by intramuscular injection. Thus, for example, the composition of the invention may be formulated with suitable polymeric or hydrophobic materials (for example as an emulsion in an acceptable oil) or ion exchange resins, or as sparingly soluble derivatives, for example, as a sparingly soluble salt.

It is more preferred that tonabersat or an analogue of formula (I) is administered in the form of a unit-dose composition for administration into a temporary or permanent human or other animal bodily orifice, such as the trachea, nostril, nasal passage, rectum, udder duct, urethra or vagina, or a surgical would, e.g. an incision, or any device inserted in such a temporary or permanent orifice, such as a catheter, trochar, cannula, endotracheal or other endoscopic tube or an ostomy tube, e.g. a tracheostomy or colostomy tube. Intranasal administration is greatly preferred

Intranasally mucosal administrable compositions may be in the form of intranasal mucosal solid compositions, such as powders and granules. They may also be in the form of intranasal mucosal fluid preparations, including liquid preparations, such as aqueous or oily blends, mixtures, suspensions, solutions, emulsions and elixirs, and gel preparations. They may also be presented as dilutable fluid concentrates or dry product reconstitutable powders for dilution or reconstitution with water or other suitable vehicle before use.

Tonabersat or an analogue of formula I, may be administered intranasally or by inhalation, typically in the form of a dry powder from a dry powder inhaler (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

Solid forms for intranasal mucosal administration are usually presented in a unit dose, and contain conventional additives, such as adjuvants, diluents, dispersing agents, excipients, colorants, desiccants, humectants, and wetting agents.

Powders and granules may be coated according to well known methods in the art. Intranasal mucosal solid formulations also include conventional sustained release formulations, such as powders or granules having a resistant coating.

Suitable excipients include cellulose, mannitol, lactose, chitosan, pectin, cellulose derivatives such as hydroxypropylmethylcellulose, methyl cellulose, hydroxyethylcellulose, carboxymethyl cellulose, polyoxamers, such as poly(ethylene oxides), gelatin, polyvinylpyrrolidone and starch. Suitable pharmaceutically acceptable wetting agents include sodium lauryl sulphate.

Solid intranasal mucosal compositions are prepared by admixture, and may be prepared by conventional methods of blending or the like. Repeated blending operations may be used to distribute the active agent throughout those compositions employing large quantities of excipients. Such operations are, of course, conventional in the art.

Intranasal mucosal fluid preparations, including gels and liquid preparations may contain conventional additives such as suspending agents, for example sorbitol, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional colouring agents.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise an analogue of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Tonabersat or an analogue of formula I, may also be administered rectally or vaginally, for example, in the form of a suppository, pessary, or enema.

Tonabersat or an analogue of formula I, may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compositions of tonabersat or an analogue of formula I, may also be in the form of fast-dispersing dosage forms such as those described in Expert Opinion in Therapeutic Patents, 11 (6), 981-986, by Liang and Chen (2001) and Verma R K et. al. Current Status of Drug Delivery Technologies and Future Directions, Pharmaceutical Technology On-Line, 2001, 25(2), 1-14. Such dosage forms are also known as oral fast-dissolving, rapid-dissolve, rapid-melt, mouth-dissolving and fast-disintegrating tablet. The composition may be in solid form which melts on contact with the tongue of the patient, for example in the form of disintegrating tablets sold under the trade name ZYDIS® (RP Scherer, UK). Alternatively, the composition may be in the form of the EFVDAS (effervescent drug absorption system, Elan Corporation), Fast Melt (highly porous microfine matrix tablet, Elan Corporation), Flashdose (floss matrix utilising shearform technology, (Fuisz Technologies, USA), Flashtab (orodispersible multiparticulate tablet, Prographarm, France), Multiflash (fast disintegrating multi-unit, multiparticulate tablet, Prographarm), Orasolv (effervescent dispersed microcapsule tablet, Cima Labs Inc, USA), Wowtab tablets (Yamanouchi Pharma Technologies, USA), LYOC (freeze dried fast dispersing tablets, Farmalyoc, France) or Quicksolve (freeze dried fast dispersing tablets, Janssen Pharamceutica, USA).

Other suitable formulation technologies may include INDAS (insoluble drug absorption system, Elan Corporation), which utilises a stabilised amorphous form of the drug with enhanced solubility, NanoCrystal technology (Elan Corporation), which utilises nanoparticles of the drug, typically having a particle size of less than 400 nm in diameter, or SoftGel (RP Scherer), which utilises a soft gelatin capsule formulation.

The formulation technologies described herein may advantageously provide more rapid drug dissolution and absorption. For those compositions that disintegrate in the oral cavity, such as beneath the tongue, the rate of absorption may be increased and first-pass metabolism effects reduced.

As is common practice, the compositions will usually be accompanied by written or printed directions for use in the medical treatment concerned.

The compositions for use in the invention may contain from 0.1% to 99% by weight, preferably from 1%-60% by weight, of the active material, depending on the method of administration.

The following are given by way of example only to illustrate and aid understanding of the invention:

Studies with tonabersat have employed a number of different formulations including:
Direct compression tablets 0.05, 1.0, 10 and 25 mg with tablet core weight 250 mg
Direct compression tablets 15, 25, 40 and 80 mg with tablet core weight 400 mg
Direct compression tablets 20 mg with core weight 400 mg
Nanoparticulate tablets 10, 20 and 40 mg with tablet core weight 400 mg

The direct compression tablets utilise micronized drug substance whilst the nanoparticulate tablets were direct compression tablets utilising wet bed milled spray dried nanoparticulate drug substance. Clinical trials have been conducted utilising 10, 20, 30, 40, 60 and 80 mg round white uncoated direct compression tablets with a core weight of 400 mg with the following unit composition (20 mg tablet only presented; all other strengths differ only in tonabersat and lactose content):

A representative formulation suitable for use in the present invention is detailed in Table 1.

TABLE 1

| | 20 mg Tablet |
|---|---|
| Ingredient | Quantity (mg) |
| Tonabersat | 20.0 |
| Lactose | 330.0 |
| Microcrystalline Cellulose | 20.0 |
| Sodium Starch Glycollate, | 24.0 |
| Type A | |
| Colloidal Silicon Dioxide | 2.0 |
| Magnesium Stearate | 4.0 |
| Total Weight | 400.0 |

### EXPERIMENTAL METHODS

To investigate the cellular events within trigeminal ganglia and determine the effect of tonabersat on neuron-satellite glia communication.

Sprague Dawley rats injected with True Blue were used to localize neuronal cell bodies in the ganglion and study neuron-glia signalling via gap junctions in the trigeminal ganglion. Dye coupling studies were conducted under basal conditions and in response to tumour necrosis factor-α injection into the whisker pad and/or capsaicin injection into the eyebrow. Changes in connexin 26 and active p38 levels were determined by immunohistochemistry.

In addition, the effect of tonabersat prior to chemical stimulation on gap junction activity and expression of connexins and active p38 was investigated.

### Animals

The animal care and procedures were conducted in accordance with institutional and National Institutes of Health guidelines. Sprague Dawley rats (Charles River Laboratories Inc., Wilmington, Mass.) were housed in clean plastic cages on a 12-hour light/dark cycle with unrestricted access to food and water.

### Dye Coupling Studies

Young male rats (200-225 g) were brought to the lab and kept in a quiet environment for about 30 minutes. Rats were anesthetized by an intraperitoneal (i.p.) injection of 0.3 ml of ketamine and xylazine solution (800 mg and 60 mg in 10 ml, respectively; Sigma, St. Louis, Mo.). Rats were observed for about 15 minutes and were assessed for effects of anesthesia using writhing reflex and tonicity of the tail (tail flick reflex). Initially, rats were injected with a fluorescent dye, True Blue (25 μl, 2 mg/ml in dimethyl sulfoxide, DMSO; Invitrogen, Eugene, Oreg.) in the whisker pad or eyebrow regions to retrogradely label neuronal cell bodies within the V1 and V2 regions of the trigeminal ganglion. Bilateral injections were performed using a 50 μL Hamilton syringe (Hamilton Company, Reno, Nev.) and a 261/2 G needle (Becton Dickinson, FranklinLakes, N.J.). Initially, dye was injected subcutaneously into both right and left whisker pads (25 μl total volume; 4 injections per side) and also injected in the brow of each eye (25 μl total; 3 injections per side). After 5 days, the animal was sacrificed and both trigeminal ganglia removed and immediately placed in Neg 50 mounting medium (Richard-Allan Scientific, Kalamazoo, Mich.) at −25° C. for further analysis. The entire ganglion was sectioned from the dorsal to ventral surface in 20 μm tissue sections using a cryostat (Microm HM525, Richard Allan Scientific) set at −25° C. Up to six sections were placed on each Superfrost Plus microscope slide (Fischer Scientific, Pittsburgh, Pa.). After fixing and permeabilizing, tissues were covered in Vectashield mounting medium (H-1000, Vector Laboratories, Burlingame, Calif.), and images (40×, 100×, or 400×) were collected using an Olympus DP70 camera mounted on an Olympus BX41 fluorescent microscope and image analysis performed using Olympus MicroSuite Five image processing software (Olympus, Center Valley, Pa.). Multiple image alignment was utilized to view the entire ganglion in a single image at 40× magnification as described previously (Thalakoti S. et. al., Neuron-Glia signalling in trigeminal ganglion: Implications for migraine pathology. Headache, 2007, 47, 1008-1023). For the gap junction studies, True Blue was bilaterally injected into the right and left whisker pads and eyebrows 5 days prior to injection of chemical agents to allow for retrograde transport of the dye to the neuronal cell bodies in the V1 and V2 regions of the ganglion. Both ganglia were then collected from untreated animals or animals injected with TNF-α (1 ng/ml in phosphate buffered saline, pH 7.4) in whisker pads for 120 minutes or injection of capsaicin (1 nM in 100% DMSO) for 15 or 60 minutes and processed for microscopic analysis as described above. For the TNF-α/capsaicin study, animals were initially injected with TNF-α. After 2 hours, animals were then injected with capsaicin and ganglia collected 15 or 60 minutes following capsaicin treatment. For the tonabersat studies, animals were injected (i.p.) with 10 mg/kg ton a be rs at (cis-(−)-6-acetyl-4S-(3-chloro-4-fluorobenzoylamino)-3,4-dihydro-2,2-dimethyl-2H-1-benzopyran-3S-ol; kindly provided by Minster Pharmaceuticals) dissolved in 100% DMSO or an equal volume of DMSO 5 minutes prior to TNF-α injection. Each experimental condition was repeated in at least 3 independent experiments.

### Immunohistochemistry

Ganglia were collected from untreated animals or animals injected with TNF-α, capsaicin, or TNF-α and capsaicin as described above. Sections were fixed using 4% paraformaldehyde for 60 minutes, permeabilized with 0.3% Triton-X100 for 60 minutes, and then incubated in 5% donkey serum for another 60 minutes. Sections were then incubated overnight at 4° C. with primary antibodies against connexin 26 (1:500 in PBS; Chemicon, Temecula, Calif.) or active p38 MAP kinase (1:200; Cell Signaling Technology, Danvers, Mass.). Immunoreactive proteins were visualized following incubation with Rhodamine Red Xconjugated donkey anti-rabbit antibodies (diluted 1:100 in PBS, Jackson Immuno-Research Laboratories, West Grove, Pa.) for 1 hour at room temperature and then mounted in Vectashield medium (H-1200) containing 4′,6 diamidino-2-phenylindole (DAPI) to allow for identification of neuronal and glial cell nuclei. As a control, some sections were incubated for 1 hour at room temperature with only the Rhodamine Red Xconjugated anti-rabbit antibodies. Images were collected as described above.

### Results

To determine whether tonabersat could inhibit the increased gap junction activity seen in response to TNF-α and capsaicin, dye coupling studies and fluorescent microscopy were used. For these studies, True Blue was injected into both the whisker pads and eyebrows and after 5 days the trigeminal ganglia were obtained from animals that were either unstimulated (control) or were injected with 1 ng/ml TNF-α for 2 hours, 1 nM capsaicin for 60 minutes, first with TNF-α and then 2 hours later with capsaicin, or with 10 mg/kg tonabersat (i.p.) prior to the TNF-α and capsaicin injections. This amount of tonabersat was selected because this dose has been shown in animal models to block trigeminal nerve activation (Parsons A. et. al., Tonabersat (SB-220453) a novel benzopyran with anticonvulsant properties attenuates trigeminal nerve-induced neurovascular reflexes, Brit. J. Pharmacol. 2001, 132, 1549-1557). In ganglia from control animals, the fluorescent dye was observed primarily in neuronal cell bodies that were localized within the V1 and V2 regions. Similarly, the dye was localized predominantly in neuronal cell bodies in both regions of the ganglia from TNF-α or capsaicin injected animals. The number of labelled satellite glial cells in the V1 region was greater after TNF-α and capsaicin treatment when compared to control, TNF-α, or capsaicin treated ganglia. Pre-treatment of animals with tonabersat greatly reduced stimulated dye movement between neurons and satellite glial cells in both the V1 and V2 regions of ganglia obtained from animals injected initially with TNF-α and then capsaicin. These data provide the first evidence to our knowledge that tonabersat can function to block gap junction signalling.

To determine whether the inhibitory effect of tonabersat on gap junction communication might involve changes in connexin 26 expression, ganglia were obtained from animals that were either unstimulated or were injected with 1 ng/ml TNF-α for 2 hours, 1 nM capsaicin for 60 minutes, first with TNF-α and then 2 hours later with capsaicin for an additional 60 minutes, or with 10 mg/kg tonabersat (i.p.) prior to the TNF-α and capsaicin injections. The intensity and pattern of connexin 26 staining was similar in control ganglia and ganglia obtained from TNF-α or capsaicin treated animals. Only low level connexin 26 expression was observed in the V1 and V2 regions. Interestingly, pre-treatment of animals with tonabersat greatly decreased the level of connexion 26 staining in V1 and V2 regions compared to animals treated with TNF-α and capsaicin. As seen at higher magnification, not only was the intensity of connexin 26 greatly reduced by tonabersat pre-treatment but the number of plaques observed between neurons and satellite glial cells in ganglia obtained from TNF-α and capsaicin treated animals was much lower. These data provide evidence that tonabersat inhibits stimulated dye coupling and hence gap junction communication between neurons and satellite glial cells by downregulation of connexin 26 levels and decreasing the number of gap junction plaques.

To determine whether TNF-α sensitization of capsaicin-responsive neurons could lead to increased expression of p38, animals were injected with TNF-α, capsaicin, or both agents and ganglion sections stained for the active phosphorylated form of p38. A low level of active p38 was observed in the V1 and V2 regions of ganglia obtained from unstimulated control animals or animals treated with TNF-α or capsaicin. In contrast, the level of p38 was greatly increased in all the neuronal bands located in the V1 and V2 regions of the ganglion in response to TNF-α and capsaicin. Importantly, a greater amount of active p38 staining was localized in the nucleus of neuronal and satellite glial cells in ganglia of animals treated with both TNF-α and capsaicin when compared to control or TNF-α or capsaicin only treated animals. Pre-treatment of animals with tonabersat greatly decreased active p38 levels and the amount of nuclear staining in both neurons and satellite glial cells in V1 and V2 regions when compared to animals treated with both TNF-α and capsaicin. Tonabersat treatment lowered p38 levels to those observed in ganglia obtained from untreated control animals or animals injected with only TNF-α or capsaicin. These results demonstrate that tonabersat greatly inhibits the level of active p38 in trigeminal ganglion neurons and satellite glia in response TNF-α and capsaicin.

1. Tonabersat or an analogue of formula 1 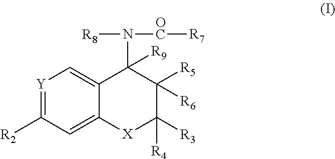
Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₉ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₃—N—CO—R₇ group being cis to the R₅ group;
and X is oxygen or NR₁₂ where R₁₂ is hydrogen or C₁₋₆ alkyl or a pharmaceutically acceptable composition thereof, for use in the treatment or prevention of a disorder selected from trigeminal neuralgia associated with intracranial tumors, rhinitis and/or sinusitis, including chronic rhinitis, inflammatory disorders, multiple myeloma, rheumatoid arthritis, autoimmune disease, chronic inflammation, acute inflammation, hypersensitivity reaction, asthma, wound healing, tissue repair, skin disorders, psoriasis, hyperkeratosis disorders, such as Vohwinkel syndrome, keratitis-ichthyosis-deafness syndrome, palmoplantarkeratoderma with deafness, hystrix-like ichthyosis with deafness, keratopachydermia and constrictions of fingers and toes with deafness.

2. (canceled)

3. Tonabersat or an analogue of formula 1 according to claim 1, wherein the disorder is one where downregulation of connexin 26 and/or p38 expression may be beneficial.

4. Tonabersat or an analogue of formula 1 according to claim 3 wherein downregulation of connexin 26 and/or p38 expression in either the V1 and/or V2 regions of trigeminal ganglia may be beneficial.

5. Tonabersat or an analogue of formula 1 according to claim 1 wherein the disorder is
trigeminal neuralgia associated with intracranial tumors, where the intercranial tumor is vestibular schwannoma.

6. A method for the treatment or prevention of a disorder selected from trigeminal neuralgia associated with intracranial tumors, rhinitis and/or sinusitis, including chronic rhinitis, inflammatory disorders, multiple myeloma, rheumatoid arthritis, pain, autoimmune disease, chronic inflammation, acute inflammation, hypersensitivity reaction, asthma, wound healing, tissue repair, skin disorders, psoriasis, hyperkeratosis disorders, such as Vohwinkel syndrome, keratitis-ichthyosis-deafness syndrome, palmoplantarkeratoderma with deafness, hystrix-like ichthyosis with deafness, keratopachydermia and constrictions of fingers and toes with deafness comprising administering to a patient in need thereof a pharmaceutically effective amount of tonabersat or an analogue of formula 1, 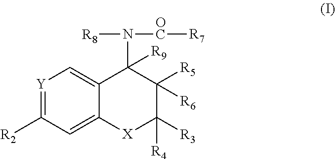
Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₈ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl;
the R₈—N—CO—R₇ group being cis to the R₅ group;
and X is oxygen or NR₁₂ where R₁₂ is hydrogen or C₁₋₆ alkyl or a pharmaceutically acceptable composition thereof.

7. A method according to claim 6, wherein the disorder is one where downregulation of connexin 26 and/or p38 expression may be beneficial.

8. A method according to claim 7 wherein downregulation of connexin 26 and/or p38 expression in either the V1 and/or V2 regions of trigeminal ganglia may be beneficial.

9. A method according to claim 6 wherein the disorder is selected from
rhinitis and/or sinusitis, including chronic rhinitis.

10. (canceled)
